(19) United States
(12) Patent Application Publication
(10) Pub. No.: US 2008/0097399 A1
(43) Pub. Date: Apr. 24, 2008

(54) Catheter With Adjustable Stiffness

(76) Inventors: Ravish Sachar, Raleigh, NC (US); Glenn M. Walker, Cary, NC (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/763,929

(22) Filed: Jun. 15, 2007

Related U.S. Application Data
(60) Provisional application No. 60/813,920, filed on Jun. 15, 2006.

(51) Int. Cl. A61M 25/00

(52) U.S. Cl. 604/525

(57) ABSTRACT

A catheter has a section of the catheter shaft adjacent its proximal end in which an outer catheter wall and an inner catheter wall define a space therebetween. An electrode is disposed within the space defined between the inner and outer catheter walls. A magnetorheological fluid fills the space between the inner and outer catheter walls. When an electric current is passed through the electrode, the magnetorheological fluid stiffens, causing the section of the catheter shaft adjacent the proximal end to stiffen.

### TECHNICAL FIELD

The present invention relates generally to catheters and relates more specifically to a catheter having a shaft with adjustable stiffness.

### BACKGROUND OF THE INVENTION

Stents are an increasingly popular treatment modality for stenosed blood vessels within the body via minimally invasive techniques. Stenting procedures for cardiac vessels are well established. However, procedures for percutaneously treating carotid, and especially intracranial, stenoses are still evolving. There are several unmet needs for the treatment of carotid and intracranial atherosclerosis. One fundamental problem is that, in order to deliver stents to the appropriate location in the carotid artery or in the intracranial circulation, the catheter requires a certain degree of stiffness. However, stiff catheters are difficult to navigate through the tortuous vessels characteristic of the carotid arteries and intracranial vessels. Thus, in order to position a stiff catheter for stent delivery, multiple guide wires and catheters must be interchanged inside the patient before a catheter of appropriate stiffness is in place, adding to the possibility of complications. Such catheter exchanges are necessary not only in carotid and intracranial procedures, but also in other vascular beds. For example, treatment of superficial femoral artery stenoses often requires a contralateral approach with multiple catheter exchanges, which increases procedural time and risk, and further increases the radiation exposure to the operator.

Ideally, a catheter should be compliant during insertion to navigate the tortuous vessels. Once positioned, the catheter should be stiff enough to handle the forces imparted on it during stent delivery, or else the catheter will move from the lesion site.

However, all prior research has been focused on two areas: imaging and actuation. Technology has been researched that would allow physicians to visualize the operative site at the tip of the catheter by adding imaging elements to the catheter. Methods of steering a catheter have been investigated, relying mainly on shape memory alloys (SMAs) that change their shape when an electric current is applied. Other novel improvements include the ability to “feel” the blood vessel surface via sensors on the catheter tip. The application proposed here, that of using MEMS to change the catheter stiffness, and not its position or shape, is a novel concept.

Catheters are used as conduits to guide therapy, such as stents, to lesion sites within the body. Without a catheter, the stent would cause tremendous damage to blood vessel walls as it scraped them en route to the site of a stenosis. In order to position a sufficiently large and stiff catheter next to a stenosis, the operator first advances a guide wire, via a leg or arm artery, up to the lesion and then slides a catheter over the wire to the lesion site. A series of steps, outlined in FIG. 1, are then performed to position a sufficiently stiff catheter for stent delivery.

There are two major problems with current endovascular procedures. First, a stiff guide catheter or sheath is usually necessary for delivering stents to the lesion site. Compliant catheters slide back, or prolapse, as stents are pushed through them. This necessitates the use of stiff catheters. However, such stiff catheters are difficult to negotiate through tortuous vessels, thus limiting the ability to position the catheter near the lesion site. This is especially true when accessing intracranial vessels for the treatment of intracranial stenoses or in the setting of acute stroke.

In order to overcome this problem, physicians use compliant catheters to access a lesion and then perform a series of exchanges with catheters of gradually increasing stiffness. Stiff catheters are often difficult to position, and increase the risk of vascular complications. Even with a stiff catheter in place, it often has to be forcefully held in place during stent delivery to prevent prolapse as the stent is advanced. The use of force during stent delivery can be a very uncomfortable experience for the patient. In the case of intracranial stent delivery, even the most pliable currently available catheters do not allow the operator to directly access a lesion, and the subsequent stent transport through the intracranial vasculature to the treatment site can be potentially dangerous to the patient.

Another drawback is that the current stent delivery procedure is very time-consuming, which translates into increased costs and hazards for the patient. At least one series of catheter and guidewire exchanges is normally required for proper positioning, and up to three can be required. Positioning the catheter near the lesion site can take up to 30% of the total procedure time. In a worst-case scenario, placing the appropriate catheter for intracranial stent delivery can take one hour or more. Each removal and insertion of a guide wire and catheter increases the chances of complications and increases radiation exposure to the patient and medical personnel. As a general rule of thumb, the length of the stenting procedure is proportional to the number and severity of complications.

An ideal catheter would be compliant during insertion to navigate tortuous vessels, and once positioned, would be stiff enough to handle the forces imparted on it by stent delivery.

The envisioned product is a smart catheter that possesses an adjustable stiffness so that it can be used in a variety of stenting procedures. This catheter will greatly simplify current procedures, saving time and money, and will also improve patient safety. Microfabrication can be used to make the catheter, which as a manufacturing technology possesses the economics of scale. Many devices can be fabricated in parallel to reduce per-device cost.

Thus there is a need for ______.

### SUMMARY OF THE INVENTION

Stated generally, the present invention relates to a catheter having a portion of its shaft adjacent the proximal end being of adjustable stiffness. A portion of the catheter shaft adjacent its proximal end has an outer catheter wall and an inner catheter wall defining a space therebetween. An electrode is disposed within the space defined between the inner and outer catheter walls. A magnetorheological fluid fills the space between the inner and outer catheter walls. When an electric current is passed through the electrode, the magnetorheological fluid stiffens, causing the section of the catheter shaft adjacent the proximal end to stiffen.

Objects, features, and advantages of the present invention will become apparent upon reading the following specification, when taken in conjunction with the drawings and the appended claims.

### BRIEF DESCRIPTION OF THE DRAWINGS

FIG. 1 is a schematic view of a catheter according to a disclosed embodiment of the present invention.

FIG. 2 is a cross-sectional view of the section 2 of FIG. 1.

FIG. 3 is a schematic view of a branch of a stenosed vessel with the catheter of FIG. 1 inserted.

FIG. 4 is a cross-sectional view of the section 4 of FIG. 3

FIG. 5 is a schematic view of the branch of the vessel of FIG. 3 with the catheter of FIG. 1 inserted and actuated.

FIG. 6 is a cross-sectional view of the section 6 of FIG. 5.

FIG. 7 is a schematic view of a planar microcoil patterned on a flexible substrate.

FIG. 8 is a schematic view of the patterned substrate of FIG. 7 formed into a cylinder.

### DETAILED DESCRIPTION OF THE DISCLOSED EMBODIMENT

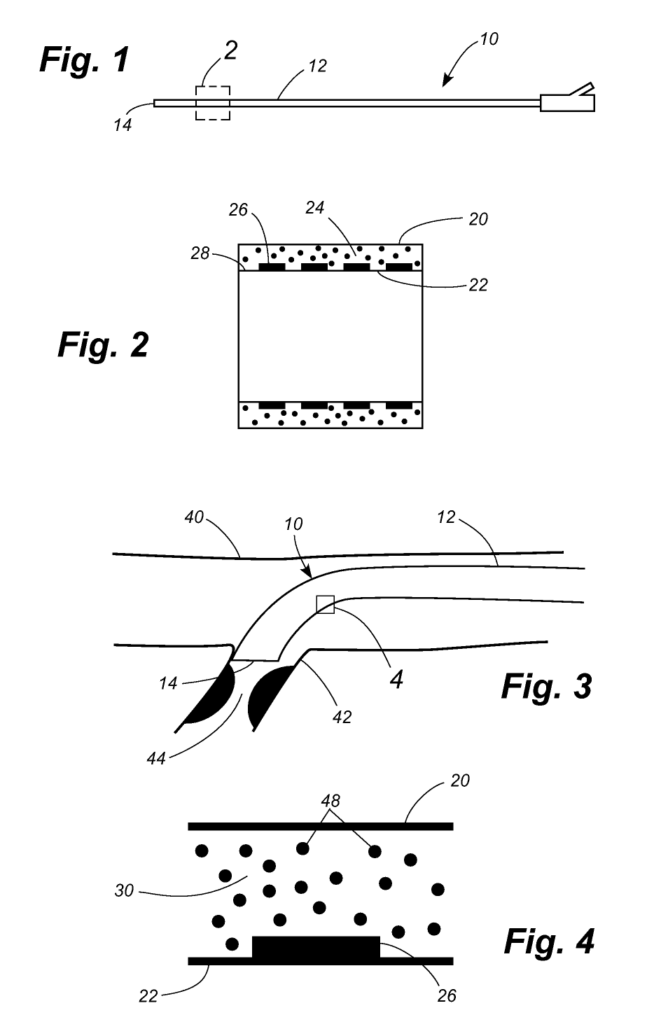

Referring now to the drawings, in which like numerals indicate like elements throughout the several views, FIG. 1 shows a catheter 10 having a shaft 12 with a proximal end 14. As shown in FIG. 2, a portion 16 of the catheter shaft 12 adjacent the proximal end 14 has an outer wall 20 and an inner wall 22. An annular space 24 is formed between the inner and outer walls 20, 22. In the disclosed embodiment the length of the catheter shaft comprising the concentric tubes is approximately six to eight inches in length. A plurality of electrode sections 26 are formed on the outer surface 28 of the inner wall 22. A magnetorheological (MR) fluid 30 fills the annular space 24 between the inner and outer walls 20, 22.

The catheter 10 is normally soft and pliable. However, when a magnetic field is applied to the MR fluid 30 in the portion of the shaft 12 adjacent the proximal end 14 of the catheter 10, that portion of the shaft stiffens, as is characteristic of MR fluids. FIG. 3 illustrates a vessel 40 having a branch 42. A stenosis 44 is formed in the branch 42. The proximal end 14 of the catheter 10 has been steered into the branch 42 to a location adjacent the stenosis 44. The naturally pliable state of the catheter 10 facilitates positioning. FIG. 4 illustrates the MR fluid in the proximal portion of the catheter shaft with the catheter in its normal, deactuated state. The magnetic particles 48 are randomly distributed throughout the MR fluid 30.

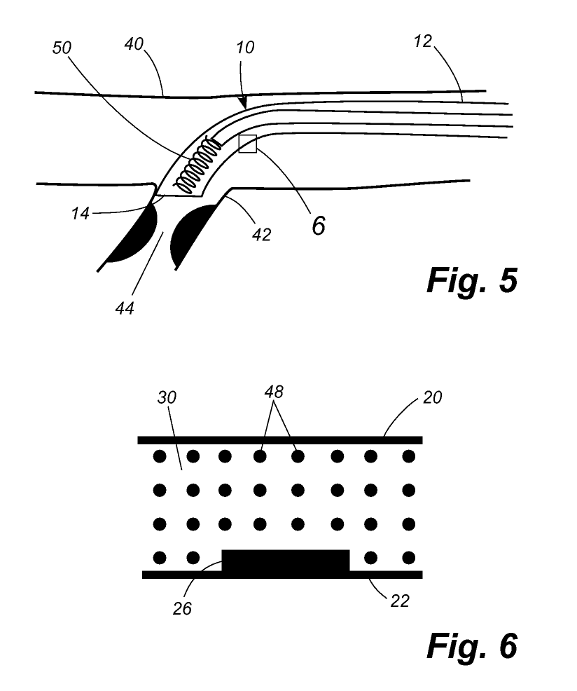

FIGS. 5 and 6 show the catheter in its actuated state. With an electric current flowing through the electrodes 26 on the outer surface 28 of the inner catheter wall 22, the magnetic particles 48 in the MR fluid 30 align, causing the fluid to stiffen. This in turn causes the proximal portion of the catheter 10 shaft to stiffen, thereby facilitating deployment of a stent 50 through the catheter shaft 12.

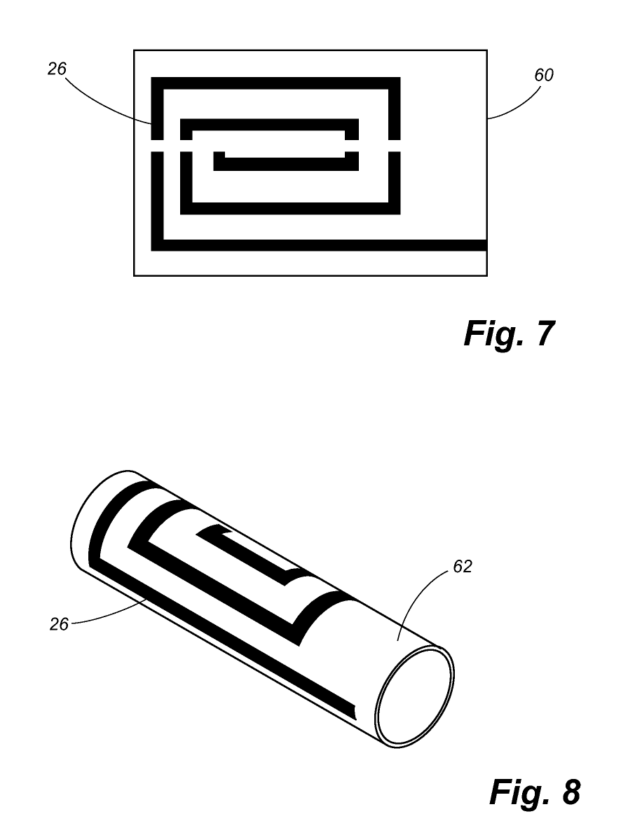

FIG. 7 shows a flexible substrate 60 having a pattern of electrodes 26 formed on its upper surface. The electrodes 26 are deposited on the flexible substrate 60 using known micro-electrical-mechanical systems (“MEMS”) technology, as will be more fully explained below. FIG. 8 shows the flexible substrate 60 formed into a cylinder 62. This cylinder is mounted within the forward end of the catheter shaft 12 and forms the inner wall 22 discussed above.

The present invention addresses the need for an improved catheter for treating vascular stenoses, especially carotid, intracranial, coronary, and lower extremity stenoses. The catheter 10 uses microfabricated electrodes and magnetorheological (MR) fluids to electronically control catheter stiffness. The smart catheter is flexible enough to be maneuvered through tortuous blood vessels and then positioned near the operative site. Once actuated, the smart catheter is stiff enough to allow the delivery of therapies, such as stents, to the lesion site. The catheter 10 overcomes current problems with catheter movement and prolapse during stent delivery. The catheter reduces the chances for injury to the patient during the procedure by reducing the number of catheters and wires that must be used and allows the catheter to be used in a variety of stenting applications.

The basic construction of the catheter is as follows: A polyimide film substrate 50 μm thick is patterned with planar spiral microelectrode geometries. Photoresist is patterned on the polyimide substrate, and a 300 Å thick adhesion layer of titanium is then sputtered followed by a 3000 Å thick layer of copper. A lift-off procedure is used to remove the photoresist, leaving the patterned electrodes. Photo-curable polyimide is then patterned on the substrate to insulate the interconnects from the external environment. The patterned substrates are then diced into pieces of an appropriate size. In the disclosed embodiment, the substrate measures 8″×0.31″. These dimensions allow the patterned substrate to be rolled up into a cylinder and then inserted into an 8 French (0.105″ I.D.) catheter. A current applied to the microelectrodes induces a magnetic field within the MR fluid, causing the MR fluid, and therefore the catheter, to stiffen.

The catheter of the present invention addresses the shortcomings of current technology by possessing both adequate compliance and adjustable stiffness. A compliant catheter is easy to position near a lesion, and once there, its stiffness can be adjusted to allow a stent to be delivered. By harnessing the power of MR fluids and microfabrication, a catheter can be made that possesses adjustable stiffness. During insertion of the catheter, no field is applied, and the fluid in the annulus is free to squeeze (flow) from one region to another as the catheter bends to navigate the blood vessels. Once the catheter is positioned, a field is applied. The field causes the suspended particles to line up in an orderly fashion. The aligned columns of particles restrict any fluid flow within the annulus. Thus, by turning the field on, the catheter walls essentially turn from a liquid to a solid.

It is important to note that enabling the field does not change the size of the catheter, nor does it move the catheter. The field simply causes the fluid within the annulus to resist shear, which effectively gives the catheter greater stiffness. Any forces the catheter experiences from advancing a stent induce a fluid flow within the catheter annulus. Because the actuated fluid resists shear, and thus fluid flow, the catheter is resistant to any motion. Stiffness can be adjusted to different degrees by modulating the field strength as needed via an external power supply.

Other advantages of the catheter of the present invention are apparent upon comparison of a typical deployment procedure using a prior art catheter against the deployment procedure of the catheter of the present invention. A typical deployment procedure using a prior art catheter comprises at least the following steps: (1) insert a first guide wire; (2) insert a first catheter; (3) remove first guide wire; (4) insert a second, stiffer guide wire; (5) remove the first catheter; (6) insert a second, stiffer catheter; and (7) repeat steps 3-6 as necessary. In contrast, the deployment procedure using the catheter of the present invention comprises only the steps of: (1) inserting a guide wire; (2) inserting the catheter; and (3) adjusting the stiffness of the catheter as needed.

Finally, it will be understood that the preferred embodiment has been disclosed by way of example, and that other modifications may occur to those skilled in the art without departing from the scope and spirit of the appended claims.

What is claimed is:

1. A catheter, comprising:
a catheter shaft having a proximal end;
a section of said catheter shaft adjacent said proximal end having an outer catheter wall and an inner catheter wall defining a space therebetween;
an electrode disposed within said space defined between said inner and outer catheter walls; and
a magnetorheological fluid disposed in said space defined between said inner and outer catheter walls,
whereby when an electric current is passed through said electrode, said magnetorheological fluid stiffens, causing said section of said catheter shaft adjacent said proximal end to stiffen.

2. The catheter of claim 1, wherein said space defined between said inner and outer catheter walls comprises an annular space.

3. The catheter of claim 1, wherein said a section of said catheter shaft adjacent said proximal end having an outer catheter wall and an inner catheter wall has a length of from approximately three to approximately ten inches.

4. The catheter of claim 3, wherein said a section of said catheter shaft adjacent said proximal end having an outer catheter wall and an inner catheter wall has a length of from approximately six to approximately eight inches.

5. The catheter of claim 1, wherein said electrode is disposed on the outer surface of said inner catheter wall.

6. A method of controlling the stiffness of a catheter, comprising the steps of:
providing a catheter having a catheter shaft in which inner and outer catheter walls define a space therebetween, said space being filled with a magnetorheological fluid; and
subjecting said magnetorheological fluid to a magnetic field,
whereby said magnetorheological fluid stiffens to impart rigidity to said catheter shaft.

7. The method of claim 6,
wherein said step of providing a catheter further comprises the step of providing a catheter having an electrode within said space; and
wherein said step of subjecting said magnetorheological fluid to a magnetic field comprises the step of passing an electric current through said electrode.
